(19) United States
(12) Patent Application Publication
(10) Pub. No.: US 2018/0242013 A1
(43) Pub. Date: Aug. 23, 2018

(54) MOTION DETERMINING APPARATUS, BODY-INSERTABLE APPARATUS, METHOD OF DETERMINING MOTION, AND COMPUTER READABLE RECORDING MEDIUM

(71) Applicant: OLYMPUS CORPORATION, Tokyo (JP)

(72) Inventor: Kazuya FURUHO, Tokyo (JP)

(73) Assignee: OLYMPUS CORPORATION, Tokyo (JP)

(21) Appl. No.: 15/955,818

(22) Filed: Apr. 18, 2018

Related U.S. Application Data
(63) Continuation of application No. PCT/JP2017/008203, filed on Mar. 1, 2017.

(30) Foreign Application Priority Data
Jun. 13, 2016 (JP) 2016-117333

(51) Int. Cl. H04N 19/53; G06T 7/00; A61B 1/04; H04N 19/573

(52) U.S. Cl. CPC H04N 19/53; H04N 19/573; A61B 1/041; G06T 7/0016

(57) ABSTRACT

A motion determining apparatus includes a processor comprising hardware. The processor is configured to: acquire first and second compressed data formed by compressing each of first and second images sequentially captured inside a subject by a body-insertable apparatus provided with an image sensor and an illumination device that performs irradiation with illumination light, and a parameter with regard to the illumination device at a time of capturing the first and second images with the image sensor; calculate a difference between a data amount of the first compressed data and a data amount of the second compressed data; compare the difference with a first threshold value; compare the parameter with at least one reference value; and determine whether a motion of the body-insertable apparatus is larger than a predetermined value based on comparison results of comparing the difference and comparing the parameter with at least one reference value.

### CROSS-REFERENCE TO RELATED APPLICATION

This application is a continuation of PCT International Application No. PCT/JP2017/008203 filed on Mar. 1, 2017 which claims the benefit of priority from Japanese Patent Application No. 2016-117333, filed on Jun. 13, 2016, the entire contents of which are incorporated herein by reference.

### BACKGROUND

The present disclosure relates to a motion determining apparatus, a body-insertable apparatus provided with the motion determining apparatus, a method of determining motions, and a computer readable recording medium.

An endoscope, which is inserted into a subject and generates in-vivo images inside the subject by capturing images inside the subject, has been known. With regard to the endoscope like this, a technique for detecting motions of the endoscope has been desired so that image blurring may be prevented. For example, the technique for detecting motions of the endoscope by comparing a data size of a compressed image with a predetermined threshold value has been known (see JP 2004-154176 A).

### SUMMARY

A motion determining apparatus according to one aspect of the present disclosure includes a processor comprising hardware, wherein the processor is configured to: acquire first and second compressed data formed by compressing each of first and second images sequentially captured inside a subject by a body-insertable apparatus provided with an image sensor and an illumination device that performs irradiation with illumination light, and a parameter with regard to the illumination device at a time of capturing the first and second images with the image sensor; calculate a difference between a data amount of the first compressed data and a data amount of the second compressed data; compare the difference with a first threshold value; compare the parameter with at least one reference value; and determine whether a motion of the body-insertable apparatus is larger than a predetermined value based on comparison results of comparing the difference with a first threshold value and comparing the parameter with at least one reference value.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system according to a first embodiment.

FIG. 2 is a block diagram illustrating a functional configuration of a capsule endoscope according to the first embodiment.

FIG. 3 is a flowchart illustrating an outline of processing executed by a control unit of the capsule endoscope according to the first embodiment.

FIG. 4 is a flowchart illustrating an outline of processing executed by a control unit of a capsule endoscope according to a second embodiment.

FIG. 5 is a block diagram illustrating a functional configuration of a capsule endoscope according to a third embodiment.

FIG. 6 is a flowchart illustrating an outline of processing executed by a control unit of the capsule endoscope according to the third embodiment.

FIG. 7A is a schematic diagram illustrating a light emission amount of an illumination unit in a current frame controlled by an illumination control section of the capsule endoscope according to the third embodiment.

FIG. 7B is a schematic diagram illustrating a light emission amount of the illumination unit in a next frame controlled by the illumination control section of the capsule endoscope according to the third embodiment.

FIG. 8A is a schematic diagram illustrating a charge amount of an imaging unit in the current frame controlled by an imaging control section of the capsule endoscope according to the third embodiment.

FIG. 8B is a schematic diagram illustrating a charge amount of the imaging unit in the next frame controlled by the imaging control section of the capsule endoscope according to the third embodiment.

FIG. 9 is a flowchart illustrating an outline of processing executed by a control unit of a capsule endoscope according to a fourth embodiment.

FIG. 10 is a flowchart illustrating an outline of processing executed by a control unit of a capsule endoscope according to a fifth embodiment.

FIG. 11A is a schematic diagram illustrating a light emission amount of an illumination unit in a current frame controlled by an illumination control section of the capsule endoscope according to the fifth embodiment.

FIG. 11B is a schematic diagram illustrating a light emission amount of the illumination unit in a next frame controlled by the illumination control section of the capsule endoscope according to the fifth embodiment.

### DETAILED DESCRIPTION

Hereinafter, a capsule endoscope system provided with a capsule endoscope will be described, with reference to the attached drawings, as an example of an endoscope system according to embodiments. Note that the present disclosure is not limited by the following embodiments while the following descriptions exemplify the capsule endoscope to be orally introduced into a subject to capture images. More specifically, various capsule endoscopes may be employed for the present disclosure, such as a capsule endoscope to be orally ingested by the subject with normal saline or water, for example, to capture images inside a body cavity of the subject. Moreover, in the following descriptions, the drawings merely schematically illustrate shapes, sizes, and positional relations to the extent that contents are understandable. Accordingly, the present disclosure is not limited only to the shapes, sizes, and positional relations exemplified in the drawings. The same elements are denoted by the same reference signs throughout the drawings.

### First Embodiment

Configuration of Capsule Endoscope System

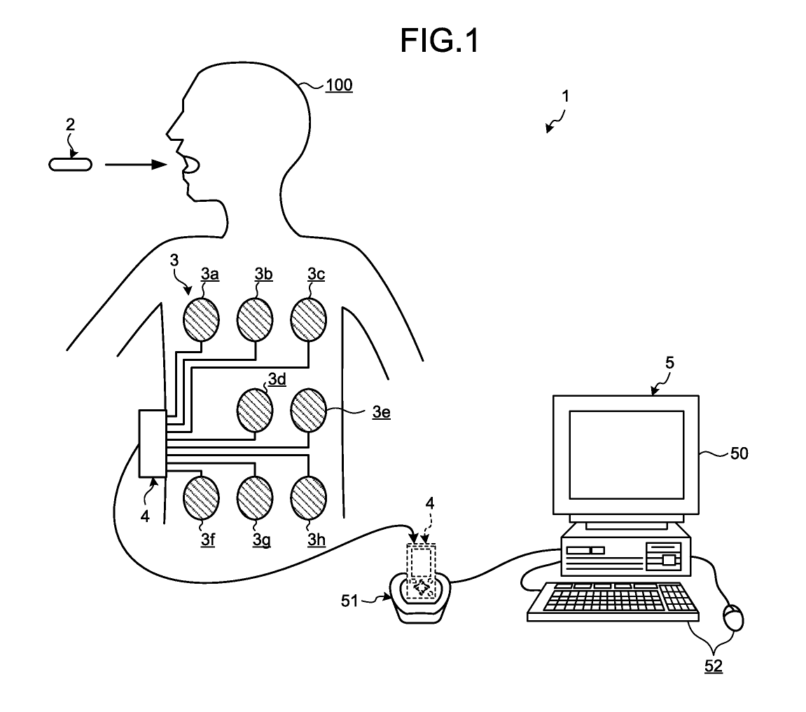

FIG. 1 is a schematic diagram illustrating a schematic configuration of a capsule endoscope system according to a first embodiment.

A capsule endoscope system 1 illustrated in FIG. 1 includes a capsule endoscope 2 that captures an in-vivo image of a subject 100, a receiving antenna unit 3 that receives a wireless signal transmitted from the capsule endoscope 2 introduced into the subject 100, a receiving device 4, and an image processing device 5. The receiving device 4, to which the receiving antenna unit 3 is detachably coupled, performs predetermined processing on the wireless signal received by the receiving antenna unit 3 to record or display the wireless signal. The image processing device 5 processes and/or displays an image corresponding to image data inside the subject 100, which is captured by the capsule endoscope 2.

The capsule endoscope 2 has an imaging function for capturing images inside the subject 100 and a wireless communication function for transmitting, to the receiving antenna unit 3, in-vivo information including the image data obtained by capturing images inside the subject 100. The capsule endoscope 2 passes through the esophagus inside the subject 100 after being swallowed by the subject 100, and moves inside a body cavity of the subject 100 by peristalsis of a digestive tract lumen. While moving inside the body cavity of the subject 100, the capsule endoscope 2 sequentially captures images inside the body cavity of the subject 100 at a minute time interval, for example, at 0.5-second intervals (2 fps), generates the image data of the images captured inside the subject 100, and transmits the image data to the receiving antenna unit 3 wirelessly and sequentially. A detailed configuration of the capsule endoscope 2 will be described later.

The receiving antenna unit 3 includes receiving antennas 3a to 3h. The receiving antennas 3a to 3h receive the wireless signal from the capsule endoscope 2 and transmit the wireless signal to the receiving device 4. The receiving antennas 3a to 3h are configured by using loop antennas. The respective receiving antennas 3a to 3h are attached at predetermined positions on an outer surface of the subject 100, for example, at positions corresponding to respective organs inside the subject 100 that are passing routes of the capsule endoscope 2.

The receiving device 4 records the image data inside the subject 100 included in the wireless signal received from the capsule endoscope 2 via the receiving antennas 3a to 3h, or displays the image corresponding to the image data inside the subject 100. The receiving device 4 records information such as position information of the capsule endoscope 2 and time information indicating time in association with the wireless signal received via the receiving antennas 3a to 3h. The receiving device 4 is housed in a receiving device holder (not illustrated) and carried by the subject 100 while examination by the capsule endoscope 2 is being performed, that is, for example, from when the capsule endoscope 2 is introduced from the mouth of the subject 100 until when the capsule endoscope 2 is discharged from the subject 100 after passing through the digestive tract. After the examination by the capsule endoscope 2 is completed, the receiving device 4 is removed from the subject 100 and coupled to the image processing device 5 to transfer the image data and the like received from the capsule endoscope 2.

The image processing device 5, configured by using a personal computer, a mobile terminal, and the like, includes a display device 50, a cradle 51, and an operation input device 52 such as a keyboard and a mouse. The display device 50 displays the image corresponding to the image data inside the subject 100 transferred from the receiving device 4. The cradle 51 reads the image data and the like from the receiving device 4. The display device 50 is configured by using a display panel employing, for example, liquid crystal or organic electro-luminescence (EL). The cradle 51 transfers, when the receiving device 4 is attached thereto, the image data, position information and time information associated with the image data, and related information such as identification information of the capsule endoscope 2 from the receiving device 4 to the image processing device 5. The operation input device 52 receives input from a user. The user diagnoses the subject 100 by observing living body regions inside the subject 100 such as esophagus, stomach, small intestine, and large intestine while operating the operation input device 52 and seeing images inside the subject 100 sequentially displayed by the image processing device 5.

Configuration of Capsule Endoscope

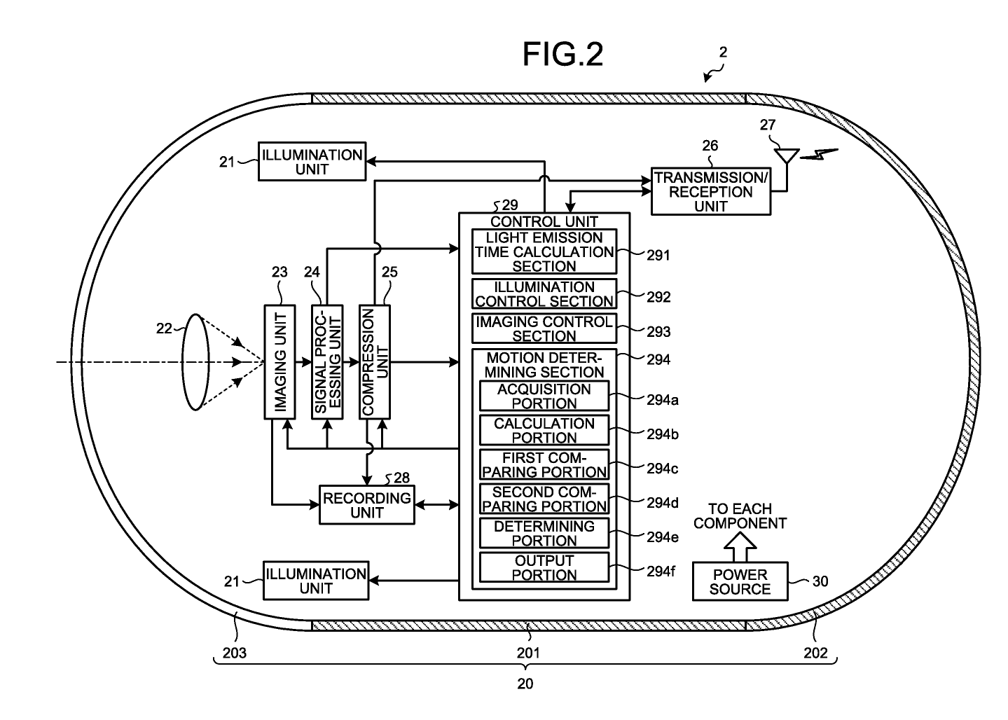

Next, the detailed configuration of the capsule endoscope 2 will be described. FIG. 2 is a block diagram illustrating a functional configuration of the capsule endoscope 2.

The capsule endoscope 2 illustrated in FIG. 2 includes an capsule-shaped casing 20, an illumination unit 21, an optical system 22 that forms a subject image, an imaging unit 23, a signal processing unit 24, a compression unit 25, a transmission/reception unit 26, a recording unit 28 that records various kinds of information of the capsule endoscope 2, a control unit 29 that controls each component of the capsule endoscope 2, and a power source 30 that supplies power to each component of the capsule endoscope 2. The capsule-shaped casing 20 is formed in a size and shape easy to introduce into the digestive tract of the subject 100. The illumination unit 21 irradiates an imaging visual field of the capsule endoscope 2 with illumination light such as white light. The imaging unit 23 generates an image signal by receiving the subject image formed by the optical system 22 and photoelectrically converting the subject image. The signal processing unit 24 generates image data by applying predetermined signal processing to the image signal generated by the imaging unit 23. The compression unit 25 compresses the image data input from the signal processing unit 24 and outputs the image data to the transmission/reception unit 26 and the control unit 29. The transmission/reception unit 26 transmits the image data input from the compression unit 25 to the outside via an antenna 27 or receives the wireless signal from the outside.

The capsule-shaped casing 20 is an outer casing formed in a size and shape capable of being introduced into organs of the subject 100, and is implemented by closing both opening ends of a cylindrical casing 201 with dome-shaped casings 202 and 203. The dome-shaped casing 203 is formed of a transparent member capable of transmitting the illumination light with which the illumination unit 21 performs irradiation. As illustrated in FIG. 2, the capsule-shaped casing 20 formed by the cylindrical casing 201 and the dome-shaped casings 202 and 203 includes the illumination unit 21, the optical system 22, the imaging unit 23, the signal processing unit 24, the compression unit 25, the transmission/reception unit 26, the antenna 27, the recording unit 28, the control unit 29, and the power source 30.

The illumination unit 21 irradiates, under the control of the control unit 29, an area including at least the imaging visual field of the capsule endoscope 2 with the illumination light such as white light through the dome-shaped casing 203. The illumination unit 21 is configured by using a light emitting diode (LED) or the like.

The optical system 22 condenses light reflected from mucosa of the subject 100 onto an imaging surface of the imaging unit 23 to form the subject image. The optical system 22 is configured by using one or more lenses such as a condenser lens or a focus lens.

The imaging unit 23 sequentially generates, under the control of the control unit 29, the image signal of the subject image formed by the optical system 22 in accordance with a predetermined frame rate, and outputs the generated image signal to each of the signal processing unit 24 and the recording unit 28. The imaging unit 23 is configured by using an image sensor such as a complementary metal oxide semiconductor (CMOS) or a charge coupled device (CCD).

The signal processing unit 24 generates, under the control of the control unit 29, the image data by applying the predetermined signal processing to the image signal input from the imaging unit 23 and outputs the image data to the compression unit 25 and the control unit 29. Here, the predetermined signal processing refers to processing such as gain adjustment processing or A/D conversion processing on the image signal. The signal processing unit 24 is configured by using an integrated circuit (IC), a large scale integration (LSI), an application specific integrated circuit (ASIC), or the like.

The compression unit 25 compresses the image data input from the signal processing unit 24 in accordance with predetermined compression processing to generate compressed image data (hereinafter referred to as compressed data), and outputs the compressed data to each of the transmission/reception unit 26, the recording unit 28, and the control unit 29. Here, examples of the predetermined compression processing include compression processing that takes a difference between pixel values of adjacent pixels to allocate fewer codes to where the difference is closer to zero, and compression processing of performing frequency conversion of the image data to allocate fewer codes to the signal having a lower frequency.

The transmission/reception unit 26 wirelessly and sequentially transmits the compressed data input from the compression unit 25 to the outside via the antenna 27. Specifically, the transmission/reception unit 26 generates the wireless signal by applying signal processing such as modulation to the compressed data input from the compression unit 25, and transmits the wireless signal to the outside. In addition, the transmission/reception unit 26 receives the wireless signal transmitted from the outside via the antenna 27, applies demodulation processing or the like to the wireless signal, and outputs the wireless signal to the control unit 29.

The recording unit 28 is configured by using a read only memory (ROM), a random access memory (RAM), or the like, and records various programs executed by the capsule endoscope 2, the compressed data, and various information being processed by the capsule endoscope 2.

The control unit 29 is configured by using a central processing unit (CPU) or the like. The control unit 29 controls driving of each component of the capsule endoscope 2, and also controls input and output of the signals between each of the components.

The following describes a detailed configuration of the control unit 29. The control unit 29 includes a light emission time calculation section 291, an illumination control section 292, an imaging control section 293, and a motion determining section 294.

The light emission time calculation section 291 calculates a light emission time of the illumination light with which the illumination unit 21 performs irradiation based on the image data input from the signal processing unit 24, and outputs a calculation result to the recording unit 28. For example, the light emission time calculation section 291 calculates the light emission time of the illumination light with which the illumination unit 21 performs irradiation based on an average luminance value of the image data.

The illumination control section 292 controls a light emission amount and a light emission timing of the illumination light with which the illumination unit 21 performs irradiation based on the light emission time calculated by the light emission time calculation section 291. For example, the illumination control section 292 causes the illumination unit 21 to perform irradiation with the illumination light for the light emission time calculated by the light emission time calculation section 291.

The imaging control section 293 controls each of the imaging unit 23 and the signal processing unit 24 based on a determination result of the motion determining section 294, which will be described later.

The motion determining section 294 determines a motion of the capsule endoscope 2 with respect to the subject and outputs the determination result to the imaging control section 293 and the illumination control section 292. The motion determining section 294 includes an acquisition portion 294a, a calculation portion 294b, a first comparing portion 294c, a second comparing portion 294d, a determining portion 294e, and an output portion 294f. In the first embodiment, the motion determining section 294 functions as a motion determining device.

The acquisition portion 294a acquires data amounts of first compressed data and second compressed data, which are the compressed data of first image data and second image data that are temporally adjacent to each other, and a parameter with regard to the illumination unit 21 or the imaging unit 23 at the time when the capsule endoscope 2 has captured the first image data and the second image data. Specifically, the acquisition portion 294a acquires, from the recording unit 28, data amounts of the compressed data in a previous frame and the compressed data in a current frame that are temporally adjacent to each other, and also acquires a light emission time in the current frame and a light emission time in a next frame.

The calculation portion 294b calculates a difference in the data amount between the compressed data in the previous frame and the compressed data in the current frame that are acquired by the acquisition portion 294a.

The first comparing portion 294c compares the difference in the data amount calculated by the calculation portion 294b with a first threshold value. Here, an example of the first threshold value is as follows. A data amount of the compressed data, which is the compressed image data generated by capturing a sample with the imaging unit 23 in a state where the capsule endoscope 2 is moved, is subtracted from another data amount of the compressed data, which is the compressed image data generated by capturing the sample with the imaging unit 23 in a state where the capsule endoscope 2 is fixed. Then, the calculated value is multiplied by a factor.

The second comparing portion 294d compares the parameter acquired by the acquisition portion 294a with a reference value. Here, the parameter represents the light emission time of the illumination light with which the illumination unit 21 performs irradiation. Specifically, the second comparing portion 294d determines whether the light emission time is equal to or more than a second threshold value as the reference value, or whether the light emission time is equal to or less than a third threshold value. In more detail, the parameter is a ratio between the light emission time of the illumination light with which the illumination unit 21 performs irradiation in the current frame and the light emission time of the illumination light with which the illumination unit 21 performs irradiation in the next frame. Further, the second and third threshold values represent a value as follows. A light emission time, which is based on the image data generated by capturing the sample with the imaging unit 23 in a state where the capsule endoscope 2 is fixed, is divided by another light emission time that is based on the image data generated by capturing the sample with the imaging unit 23 in a state where the capsule endoscope 2 is moved. Then, the calculated value is multiplied by a factor.

The determining portion 294e determines whether a motion of the capsule endoscope 2 is large based on a comparison result of the first comparing portion 294c as well as a comparison result of the second comparing portion 294d. Specifically, the determining portion 294e determines that the motion of the capsule endoscope 2 is large, in a case where the first comparing portion 294c has determined that the difference in the data amount calculated by the calculation portion 294b is equal to or more than the first threshold value, and the second comparing portion 294d has determined that the parameter acquired by the acquisition portion 294a is equal to or more than the second threshold value, or equal to or less than the third threshold value.

The output portion 294f outputs information indicating that the motion of the capsule endoscope 2 is large when the determining portion 294e has determined that the motion of the capsule endoscope 2 is large.

The power source 30 includes a storage battery, such as a button battery or a capacitor, a switch to be switched by a command from the control unit 29, and the like. The power source 30 supplies power to each component of the capsule endoscope 2.

Processing of Control Unit

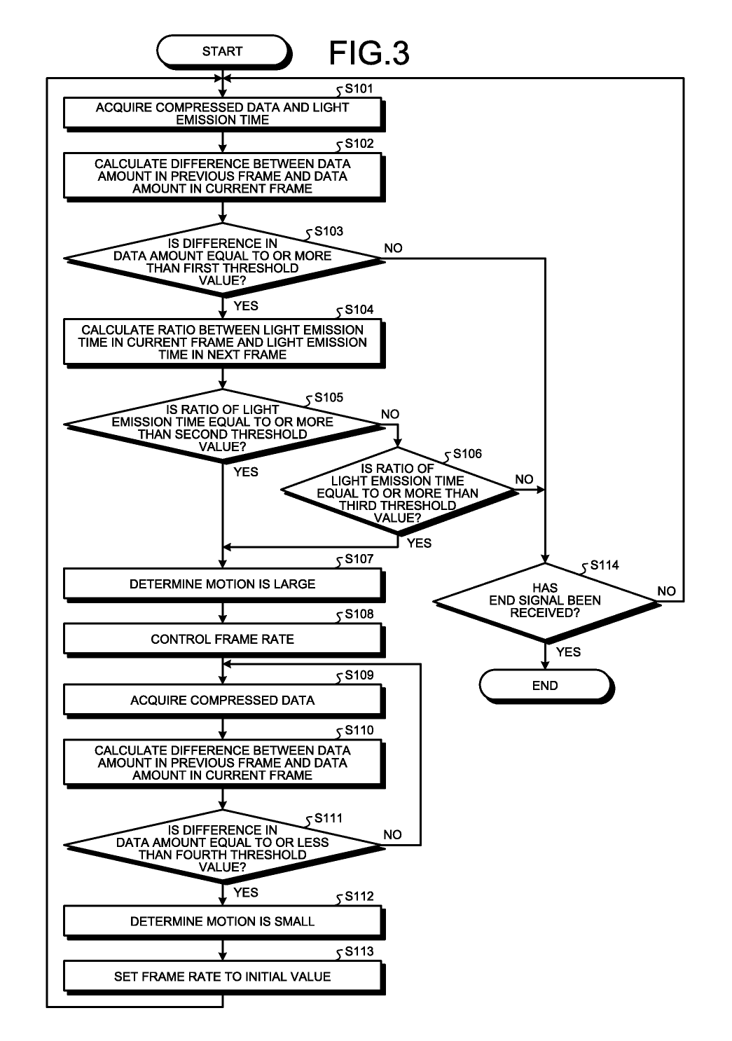

Next, processing executed by the control unit 29 will be described. FIG. 3 is a flowchart illustrating an outline of the processing executed by the control unit 29.

As illustrated in FIG. 3, first, the acquisition portion 294a acquires, from the recording unit 28, respective data amounts of the compressed data in the previous frame and the compressed data in the current frame, which are temporally adjacent to each other, and respective light emission times in the current frame and the next frame (Step S101).

Subsequently, the calculation portion 294b calculates the difference between the data amount of the compressed data in the previous frame and the data amount of the compressed data in the current frame that are acquired by the acquisition portion 294a (Step S102).

Thereafter, the first comparing portion 294c compares the difference in the data amount calculated by the calculation portion 294b with the first threshold value, and determines whether the difference in the data amount is equal to or more than the first threshold value (Step S103). When a movement amount of the capsule endoscope 2 with respect to the subject is large, a difference between pixel vales of adjacent pixels of the compressed data in the current frame becomes small due to a smoothing effect. Therefore, the data amount of the compressed data becomes smaller than in the case where a movement of the capsule endoscope 2 with respect to the subject is small or the capsule endoscope 2 is stopped. Specifically, the data amount of the compressed data is decreased due to an increased compression rate of the compression unit 25 when the movement of the capsule endoscope 2 is large, whereas the data amount is increased due to a decreased compression rate when the movement of the capsule endoscope 2 is small. In the first embodiment, the first comparing portion 294c compares the difference between the data amount of the compressed data in the previous frame and the data amount of the compressed data in the current frame with the first threshold value, and determines whether the data amount of the compressed data in the current frame is less than the data amount of the compressed data in the previous frame. When the first comparing portion 294c has determined that the difference in the data amount calculated by the calculation portion 294b is equal to or more than the first threshold value (Step S103: Yes), the control unit 29 proceeds to Step S104, which will be described later. On the other hand, when the first comparing portion 294c has determined that the difference in the data amount calculated by the calculation portion 294b is not equal to or more than the first threshold value (Step S103: No), the control unit 29 proceeds to Step S114, which will be described later.

In Step S104, the calculation portion 294b calculates a ratio between the light emission time in the current frame and the light emission time in the next frame that are acquired by the acquisition portion 294a (Step S104).

Subsequently, the second comparing portion 294d compares the ratio calculated by the calculation portion 294b with the second threshold value, and determines whether the ratio is equal to or more than the second threshold value (Step S105). The light emission time in the next frame becomes significantly longer when an acute change in a direction away from a capturing object occurs (scene change occurs) with regard to a capturing scene of the capsule endoscope 2 than in a case where the capturing scene of the capsule endoscope 2 is unchanged. Accordingly, in the first embodiment, the second comparing portion 294d compares the ratio between the light emission time in the current frame and the light emission time in the next frame with the second threshold value, and determines whether the light emission time in the next frame is significantly longer than the light emission time in the current frame. When the second comparing portion 294d has determined that the ratio calculated by the calculation portion 294b is equal to or more than the second threshold value (Step S105: Yes), the control unit 29 proceeds to Step S107, which will be described later. On the other hand, when the second comparing portion 294d has determined that the ratio calculated by the calculation portion 294b is not the second threshold value or more (Step S105: No), the control unit 29 proceeds to Step S106, which will be described later.

In Step S106, the second comparing portion 294d compares the ratio calculated by the calculation portion 294b with the third threshold value, and determines whether the ratio is equal to or less than the third threshold value. The light emission time in the next frame becomes significantly shorter when an acute change occurs (scene change occurs) in the capturing scene of the capsule endoscope 2 than in the case where the capturing scene of the capsule endoscope 2 is unchanged. Accordingly, in the first embodiment, the second comparing portion 294d compares the ratio between the light emission time in the current frame and the light emission time in the next frame with the third threshold value, and determines whether the light emission time in the next frame is significantly shorter than the light emission time in the current frame. When the second comparing portion 294d has determined that the ratio calculated by the calculation portion 294b is equal to or less than the third threshold value (Step S106: Yes), the control unit 29 proceeds to Step S107. On the other hand, when the second comparing portion 294d has determined that the ratio calculated by the calculation portion 294b is not equal to or less than the third threshold value (Step S106: No), the control unit 29 proceeds to Step S114, which will be described later.

In Step S107, the determining portion 294e determines that the motion of the capsule endoscope 2 is larger than the predetermined value based on the fact that the data amount of the compressed data in the current frame is less than the data amount of the compressed data in the previous frame, and also the light emission time in the next frame is significantly larger or shorter than the light emission time in the current frame. Furthermore, the output portion 294f outputs, to the imaging control section 293, information indicating that the motion of the capsule endoscope 2 is larger than the predetermined value.

Subsequently, the imaging control section 293 controls a frame rate of the imaging unit 23 based on an output result of the output portion 294f (Step S108).

Specifically, the imaging control section 293 increases the frame rate of the imaging unit 23. For example, the imaging control section 293 controls the frame rate of the imaging unit 23 to be increased from 2 fps to 4 fps. Accordingly, the frame rate of the imaging unit 23 is increased so that an imaging timing of the imaging unit 23 is advanced, whereby missed imaging of the subject by the capsule endoscope 2 may be prevented. Furthermore, image blurring may be prevented.

Thereafter, the acquisition portion 294a acquires a data amount of the compressed data in the latest frame from the recording unit 28 (Step S109).

Subsequently, the calculation portion 294b calculates the difference between the data amounts of the compressed data in the previous frame (such as the current frame in Step S101) and the compressed data in the current frame (such as the latest frame) that are acquired by the acquisition portion 294a (Step S110).

Thereafter, the first comparing portion 294c determines whether the difference in the data amount calculated by the calculation portion 294b is equal to or less than a fourth threshold value (Step S111). Here, the fourth threshold value is a value set in a similar manner to the first threshold value. When the first comparing portion 294c has determined that the difference in the data amount calculated by the calculation portion 294b is equal to or less than the fourth threshold value (Step S111: Yes), the control unit 29 proceeds to Step S112, which will be described later. On the other hand, when the first comparing portion 294c has determined that the difference in the data amount calculated by the calculation portion 294b is not equal to or less than the fourth threshold value (Step S111: No), the control unit 29 returns to Step S109 described above.

In Step S112, the determining portion 294e determines that the motion of the capsule endoscope 2 is smaller than the predetermined value. In this case, the output portion 294f outputs, to the imaging control section 293, information indicating that the motion of the capsule endoscope 2 is smaller than the predetermined value. Following Step S112, the control unit 29 proceeds to Step S113, which will be described later.

In Step S113, the imaging control section 293 sets the frame rate of the imaging unit 23 to an initial value. For example, the imaging control section 293 sets the frame rate of the imaging unit 23 by changing a value from 4 fps to 2 fps. Following Step S113, the control unit 29 proceeds to Step S101 described above.

In Step S114, the control unit 29 completes the processing when an end signal for terminating the examination of the subject is received from the outside via the antenna 27 and the transmission/reception unit 26 (Step S114: Yes). On the other hand, the control unit 29 returns to Step S101 described above when the end signal for terminating the examination of the subject is not received from the outside via the antenna 27 and the transmission/reception unit 26 (Step S114: No).

According to the first embodiment described above, the determining portion 294e determines that the motion of the capsule endoscope 2 is larger than the predetermined value when the difference between the data amount of the compressed data in the previous frame and the data amount of the compressed data in the current frame, which is calculated by the calculation portion 294b, is equal to or more than the first threshold value while the parameter with regard to the illumination unit 21 or the imaging unit 23 at the time of capturing the first and second image data with the capsule endoscope 2, which is acquired by the acquisition portion 294a, is equal to or more than the reference value. Therefore, a motion magnitude of the capsule endoscope 2 may be determined with a high precision.

Moreover, according to the first embodiment, motions of the capsule endoscope 2 may be determined with a high precision without separately adding a circuit or the like while the capsule endoscope 2 is required to be miniaturized and to use less power.

Note that the output result of the output portion 294f may be added to the compressed data and transmitted to the outside in the first embodiment.

### Second Embodiment

Next, a second embodiment will be described. The second embodiment has the same configurations as those of the capsule endoscope system 1 according to the first embodiment described above, and differs in processing to be executed by a control unit 29. Specifically, the second embodiment determines whether a motion of a capsule endoscope 2 is large based on a difference between data amounts of compressed data and a difference between light receiving amounts received by an imaging unit while the first embodiment described above determines whether the motion of the capsule endoscope 2 is large based on the difference between the data amounts of the compressed data and a ratio between light emission times of illumination light. Hereinafter, processing executed by a control unit of a capsule endoscope according to the second embodiment will be described. Note that the same configurations as those of the capsule endoscope system 1 according to the first embodiment described above are denoted by the same reference signs, and descriptions thereof will be omitted.

Processing of Control Unit

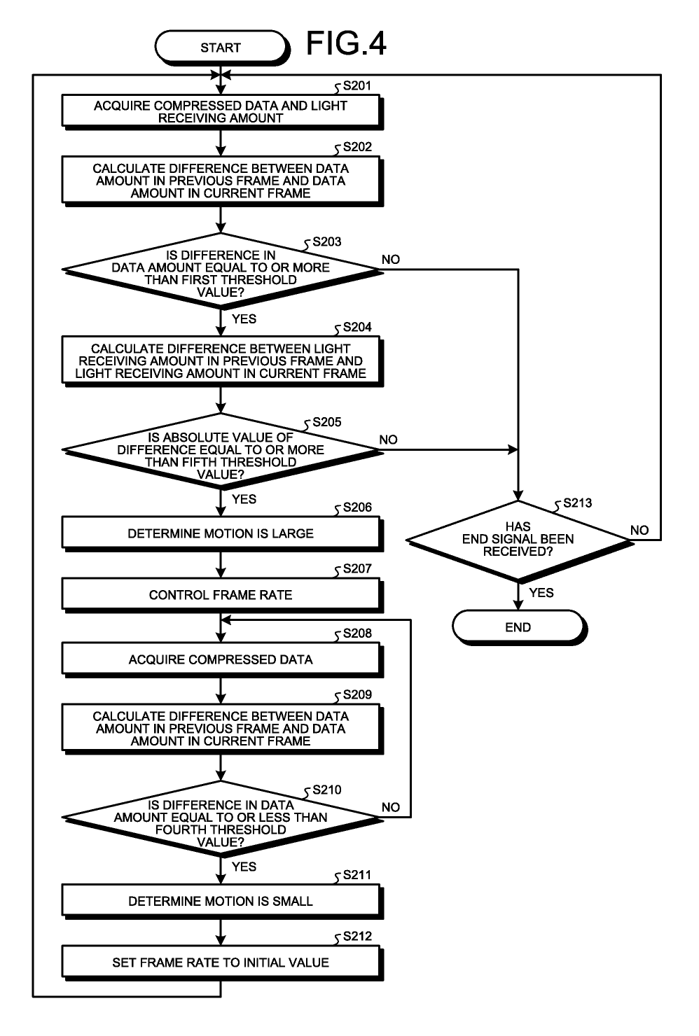

FIG. 4 is a flowchart illustrating an outline of processing executed by a control unit 29 according to the second embodiment.

As illustrated in FIG. 4, first, an acquisition portion 294a acquires, from a recording unit 28, respective data amounts of the compressed data in a previous frame and a current frame that are temporally adjacent to each other, and also acquires a light receiving amount based on image data in the previous frame and a light receiving amount based on image data in the current frame (Step S201). Here, the light receiving amount represents an average value of pixel values of the image data (luminance value).

Steps S202 and S203 correspond to Steps S102 and S103 in FIG. 3 described above, respectively.

In Step S204, a calculation portion 294b calculates the difference between the light receiving amount in the previous frame and the light receiving amount in the current frame that are acquired by the acquisition portion 294a.

Subsequently, a second comparing portion 294d determines whether an absolute value of the difference calculated by the calculation portion 294b is equal to or more than a fifth threshold value (Step S205). Here, the fifth threshold value is a value set in a similar manner to the second or third threshold value according to the first embodiment described above. When the second comparing portion 294d has determined that the absolute value of the difference calculated by the calculation portion 294b is equal to or more than the fifth threshold value (Step S205: Yes), the control unit 29 proceeds to Step S206, which will be described later. On the other hand, when the second comparing portion 294d has determined that the absolute value of the difference calculated by the calculation portion 294b is not equal to or more than the fifth threshold value (Step S205: No), the control unit 29 proceeds to Step S213, which will be described later.

Steps S206 to S213 correspond to Steps S107 to S114 in FIG. 3 described above, respectively.

According to the second embodiment described above, a determining portion 294e determines that the motion of the capsule endoscope 2 is larger than the predetermined value when the absolute value of the difference between the light receiving amount in the previous frame and the light receiving amount in the current frame is equal to or more than the fifth threshold value in a case where the difference between the data amount of the compressed data in the previous frame and the data amount of the compressed data in the current frame is determined to be equal to or more than a first threshold value. Therefore, motions of the capsule endoscope 2 may be determined with a high precision.

### Third Embodiment

Next, a third embodiment will be described. The third embodiment has different configurations as those of the capsule endoscope 2 of the capsule endoscope system 1 according to the first embodiment described above, and also differs in processing to be executed by a control unit of the capsule endoscope according to the third embodiment. Specifically, the third embodiment determines image blurring while the first embodiment described above determines motions of the capsule endoscope 2 with respect to a subject. Hereinafter, configurations of the capsule endoscope according to the third embodiment will be described. The processing executed by the control unit of the capsule endoscope according to the third embodiment will be described thereafter. Note that the same configurations as those of the capsule endoscope system 1 according to the first embodiment described above are denoted by the same reference signs, and descriptions thereof will be omitted.

Configuration of Capsule Endoscope

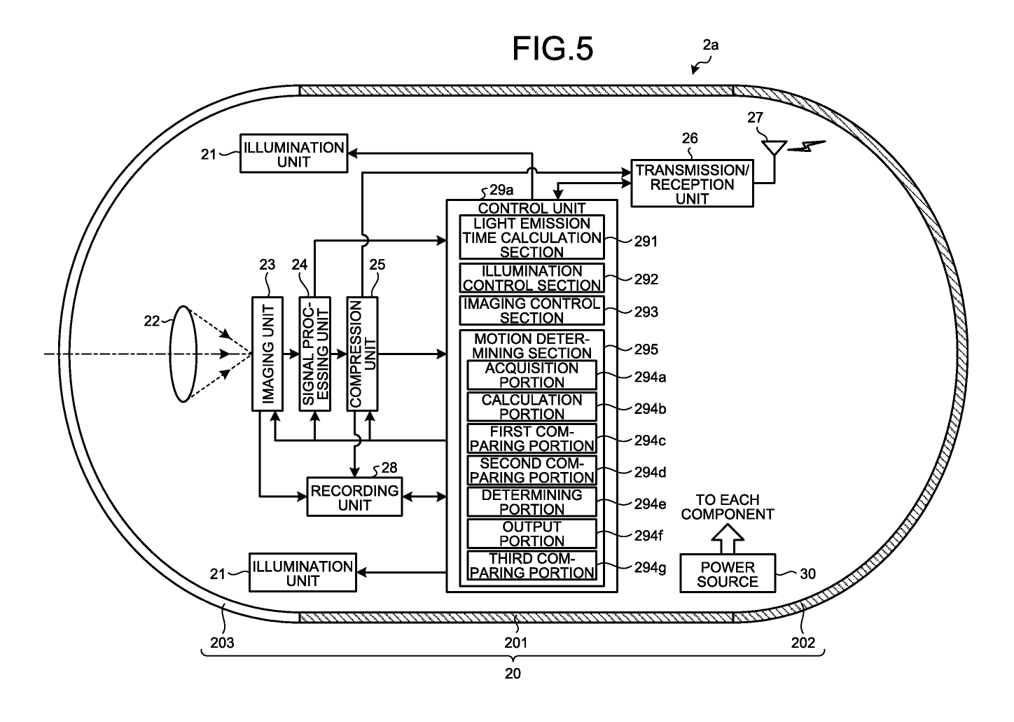

FIG. 5 is a block diagram illustrating a functional configuration of the capsule endoscope according to the third embodiment. A capsule endoscope 2a illustrated in FIG. 5 includes a control unit 29a instead of the control unit 29 of the capsule endoscope 2 according to the first embodiment described above. The control unit 29a includes a motion determining section 295 instead of the motion determining section 294 of the control unit 29 according to the first embodiment described above. Furthermore, the motion determining section 295 further includes a third comparing portion 294g in addition to the configuration of the motion determining section 294 according to the first embodiment described above.

The third comparing portion 294g compares a luminance value of a second image acquired by an acquisition portion 294a with a seventh threshold value. Specifically, the third comparing portion 294g determines whether an average pixel value of pixels of the image in a current frame acquired by the acquisition portion 294a from a recording unit 28 exceeds the seventh threshold value.

Processing of Control Unit

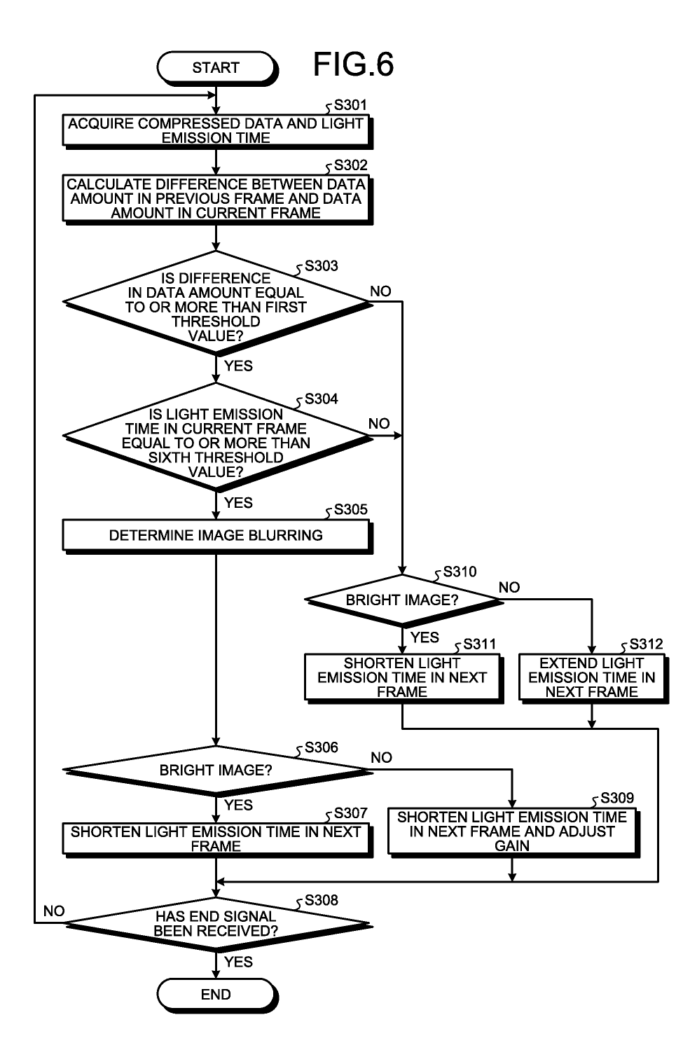

Next, processing executed by the control unit 29a will be described. FIG. 6 is a flowchart illustrating an outline of the processing executed by the control unit 29a.

In FIG. 6, Steps S301 to S303 correspond to Steps S101 to S103 in FIG. 3 described above, respectively.

In Step S304, a second comparing portion 294d determines whether a light emission time in the current frame acquired by the acquisition portion 294a is equal to or more than a sixth threshold value. When the light emission time is long, image blurring is likely to occur. Accordingly, in the third embodiment, the second comparing portion 294d determines whether the light emission time in the current frame is equal to or more than the sixth threshold value, thereby determining whether the image blurring in the current frame has occurred. Here, the sixth threshold value represents a value computed by multiplying, by a factor, the light emission time of a case where the image blurring has occurred with respect to the image data generated by capturing a sample with an imaging unit 23 in a state where the capsule endoscope 2a is moved. When the second comparing portion 294d has determined that the light emission time in the current frame is equal to or more than the sixth threshold value (Step S304: Yes), the control unit 29a proceeds to Step S305, which will be described later. On the other hand, when the second comparing portion 294d has determined that the light emission time in the current frame is not equal to or more than the sixth threshold value (Step S304: No), the control unit 29a proceeds to Step S310, which will be described later.

In Step S305, a determining portion 294e determines that the image corresponding to compressed data in the current frame is a blurred image. In this case, an output portion 294f outputs, to an illumination control section 292, information indicating that the image corresponding to the compressed data in the current frame is the blurred image.

Subsequently, the third comparing portion 294g determines whether the image data in the current frame is a bright image. Specifically, the third comparing portion 294g determines whether the average pixel value of pixels of the image data in the current frame exceeds the seventh threshold value (Step S306). When the third comparing portion 294g has determined that the image data in the current frame is the bright image (Step S306: Yes), the control unit 29a proceeds to Step S307, which will be described later. On the other hand, when the third comparing portion 294g has determined that the image data in the current frame is not the bright image (Step S306: No), the control unit 29a proceeds to Step S309, which will be described later.

In Step S307, the illumination control section 292 causes an illumination unit 21 to perform irradiation in a next frame for the light emission time that is shortened compared to the light emission time in the current frame. As a result, the image in the next frame reaches proper exposure.

In Step S308, the control unit 29a completes the processing when an end instruction signal for terminating an examination of the subject is received from the outside via an antenna 27 and a transmission/reception unit 26 (Step S308: Yes). On the other hand, the control unit 29a returns to Step S301 described above when the end instruction signal for terminating the examination of the subject is not received from the outside via the antenna 27 and the transmission/reception unit 26 (Step S308: No).

In Step S309, the control unit 29a shortens the light emission time in the next frame, and also increases a gain of a signal generated by the imaging unit 23. Specifically, the illumination control section 292 shortens the light emission time in the next frame compared to the light emission time in the current frame. Furthermore, an imaging control section 293 increases the gain of the signal generated by the imaging unit 23 so that a charge amount of the imaging unit 23 is increased. Following Step S309, the control unit 29a proceeds to Step S308.

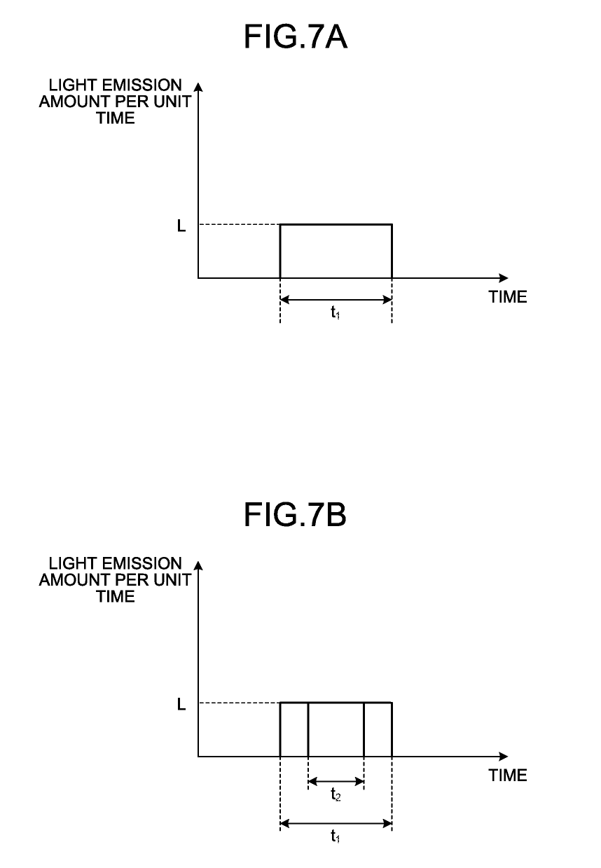

FIG. 7A is a schematic diagram illustrating the light emission amount of the illumination unit 21 in the current frame controlled by the illumination control section 292. FIG. 7B is a schematic diagram illustrating the light emission amount of the illumination unit 21 in the next frame controlled by the illumination control section 292. In FIGS. 7A and 7B, a horizontal axis represents the light emission time and a vertical axis represents the light emission amount per unit time.

As illustrated in FIGS. 7A and 7B, the illumination control section 292 shortens the light emission time in the next frame compared to the light emission time in the current frame. For example, the illumination control section 292 sets a light emission time t₂ in the next frame by shortening a light emission time t₁ in the current frame by half while maintaining the light emission amount per unit time at a light emission amount L per unit time in the current frame.

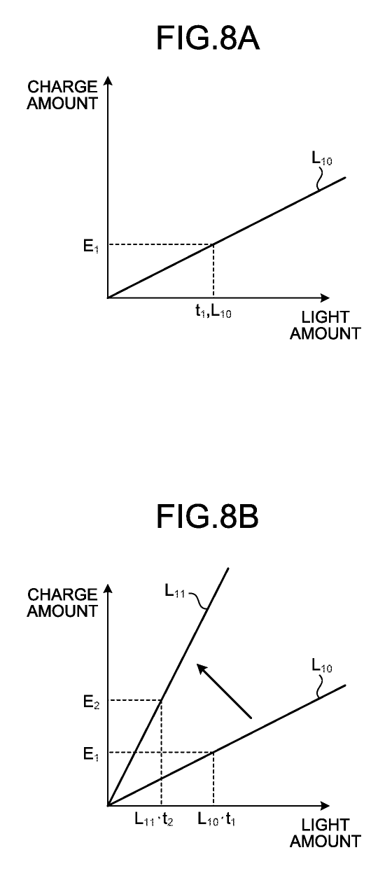

FIG. 8A is a schematic diagram illustrating the charge amount of the imaging unit 23 in the current frame controlled by the imaging control section 293. FIG. 8B is a schematic diagram illustrating the charge amount of the imaging unit 23 in the next frame controlled by the imaging control section 293. In FIGS. 8A and 8B, a horizontal axis represents a light receiving amount and a vertical axis represents the charge amount. Moreover, in FIGS. 8A and 8B, a straight line L₁₀ represents the charge amount of the imaging unit 23 in the current frame per unit amount of received light, and a straight line L₁₁ represents the charge amount of the imaging unit 23 in the next frame per unit amount of the received light.

As illustrated in FIGS. 8A and 8B, the imaging control section 293 increases the gain of the imaging unit 23 so that the charge amount of the imaging unit 23 becomes equal to or more than the charge amount in the current frame. Specifically, as illustrated by the straight line L₁₁ in FIG. 8B, the imaging control section 293 increases the gain of the signal generated by the imaging unit 23 so that a charge amount E₂ in the next frame becomes equal to or more than a charge amount E₁ in the current frame. Accordingly, the charge amount of the imaging unit 23 becomes equal to or more than the charge amount in the current frame even in a case where the light emission time in the next frame is shortened compared to the light emission time in the current frame. As a result, the light emission time of the illumination light with which the illumination unit 21 performs irradiation is shortened, whereby the image blurring may be prevented.

Step S310 and the following processing will be described with reference to FIG. 6 again.

In Step S310, the third comparing portion 294g determines whether the image data in the current frame is the bright image. When the third comparing portion 294g has determined that the image data in the current frame is the bright image (Step S310: Yes), the control unit 29a proceeds to Step S311, which will be described later. On the other hand, when the third comparing portion 294g has determined that the image data in the current frame is not the bright image (Step S310: No), the control unit 29a proceeds to Step S312, which will be described later.

In Step S311, the illumination control section 292 causes the illumination unit 21 to perform irradiation in the next frame for the light emission time that is shortened compared to the light emission time in the current frame. As a result, the image in the next frame reaches the proper exposure. Following Step S311, the control unit 29a proceeds to Step S308.

In Step S312, the illumination control section 292 causes the illumination unit 21 to perform irradiation in the next frame for the light emission time that is extended compared to the light emission time in the current frame. As a result, the image in the next frame reaches the proper exposure. Following Step S312, the control unit 29a proceeds to Step S308.

According to the third embodiment described above, when the third comparing portion 294g determines that the image data in the current frame is not the bright image, the illumination control section 292 shortens the light emission time in the next frame compared to the light emission time in the current frame while the imaging control section 293 increases the gain of the signal generated by the imaging unit 23 so that the charge amount of the imaging unit 23 is increased. Therefore, the next frame is subject to the proper exposure and the image blurring may be prevented after detecting a motion of the capsule endoscope 2a with a high precision.

Note that the gain of the signal generated by the imaging unit 23 may be increased in signal processing executed by a signal processing unit 24 while the gain is increased by the imaging control section 293 in the third embodiment.

### Fourth Embodiment

Next, a fourth embodiment will be described. The fourth embodiment has the same configurations as those of the capsule endoscope 2a according to the third embodiment described above, and differs partially in processing to be executed by a control unit. Hereinafter, the processing executed by the control unit of a capsule endoscope according to the fourth embodiment will be described. Note that the same configurations as those of the capsule endoscope system 1 according to the third embodiment described above are denoted by the same reference signs, and descriptions thereof will be omitted.

Processing of Control Unit

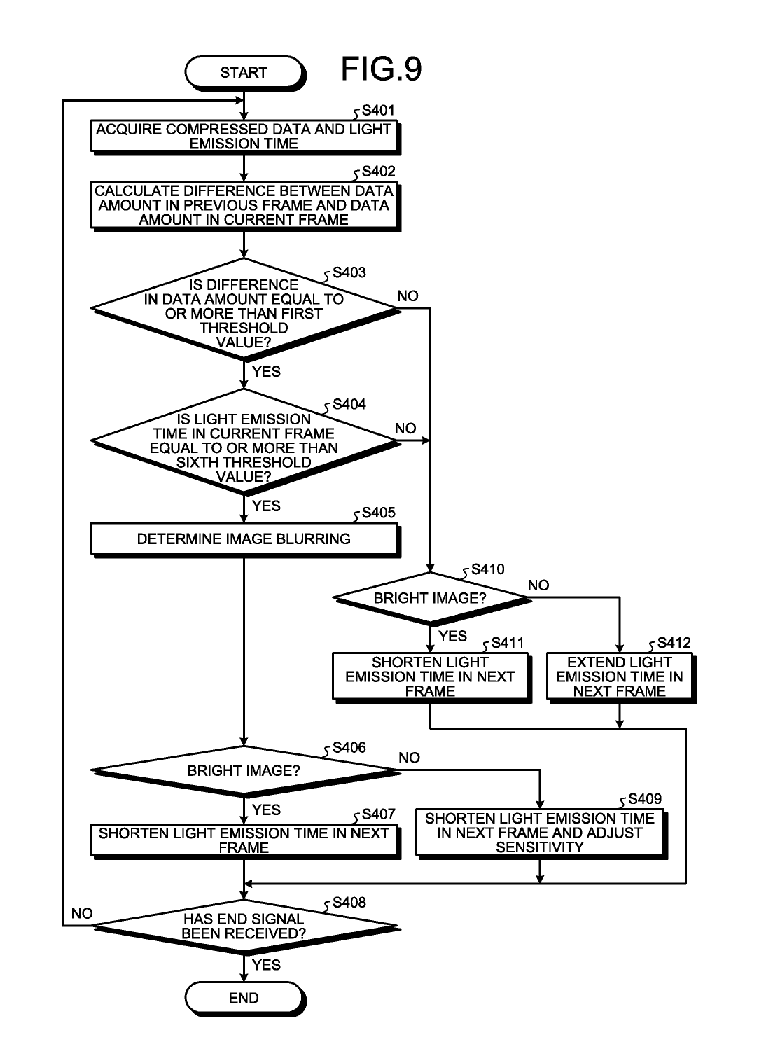

FIG. 9 is a flowchart illustrating an outline of the processing executed by a control unit 29a of the capsule endoscope 2a according to the fourth embodiment. The control unit 29a in FIG. 9 executes Step S409 instead of Step S309 in FIG. 6 described above. The other Steps S401 to S408 and Steps S410 to S412 correspond to Steps S301 to S308 and Steps S310 to S312 in FIG. 6 described above, respectively, and descriptions thereof will be omitted.

In Step S409, the control unit 29a shortens a light emission time in a next frame, and also increases sensitivity of an imaging unit 23. Specifically, the illumination control section 292 shortens the light emission time in the next frame compared to the light emission time in the current frame. Furthermore, an imaging control section 293 increases the sensitivity of the imaging unit 23 so that a charge amount of the imaging unit 23 is increased. Following Step S409, the control unit 29a proceeds to Step S408.

According to the fourth embodiment described above, when a third comparing portion 294g determines that image data in the current frame is not a bright image, the illumination control section 292 shortens the light emission time in the next frame compared to the light emission time in the current frame while the imaging control section 293 increases the sensitivity of the imaging unit 23 so that the charge amount of the imaging unit 23 is increased. Therefore, the next frame is subject to proper exposure and image blurring may be prevented after detecting a motion of the capsule endoscope 2a with a high precision.

### Fifth Embodiment

Next, a fifth embodiment will be described. The fifth embodiment has the same configurations as those of the capsule endoscope 2a according to the third embodiment described above, and differs partially in processing to be executed by a control unit. Hereinafter, the processing executed by the control unit of a capsule endoscope according to the fifth embodiment will be described. Note that the same configurations as those of the capsule endoscope system 1 according to the third embodiment described above are denoted by the same reference signs, and descriptions thereof will be omitted.

Processing of Control Unit

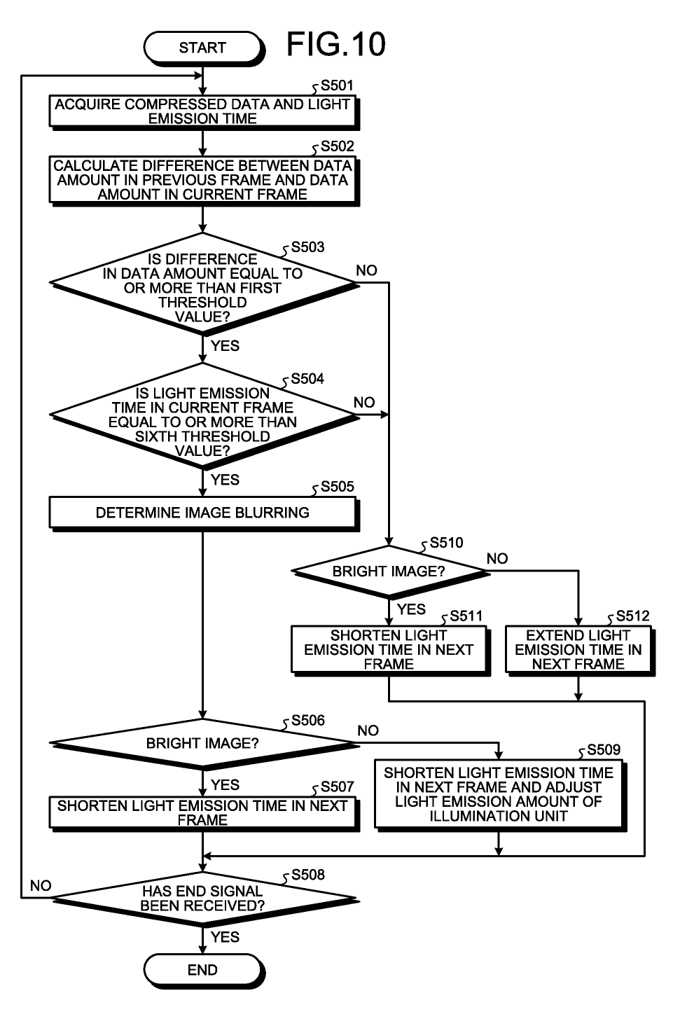

FIG. 10 is a flowchart illustrating an outline of the processing executed by a control unit 29a of the capsule endoscope 2a according to the fifth embodiment. The control unit 29a in FIG. 10 executes Step S509 instead of Step S309 in FIG. 6 described above. The other Steps S501 to S508 and Steps S510 to S512 correspond to Steps S301 to S308 and Steps S310 to S312 in FIG. 6 described above, respectively, and descriptions thereof will be omitted.

In Step S509, an illumination control section 292 shortens a light emission time in a next frame compared to the light emission time in a current frame. Besides, the illumination control section 292 performs adjustment in such a manner that a light emission amount of illumination light with which an illumination unit 21 performs irradiation in the next frame becomes larger than the light emission amount of the illumination light with which the illumination unit 21 performs irradiation in the current frame, such that a light receiving amount received by an imaging unit 23 becomes equal to or exceeds the light receiving amount in the current frame. Following Step S509, the control unit 29a proceeds to Step S508.

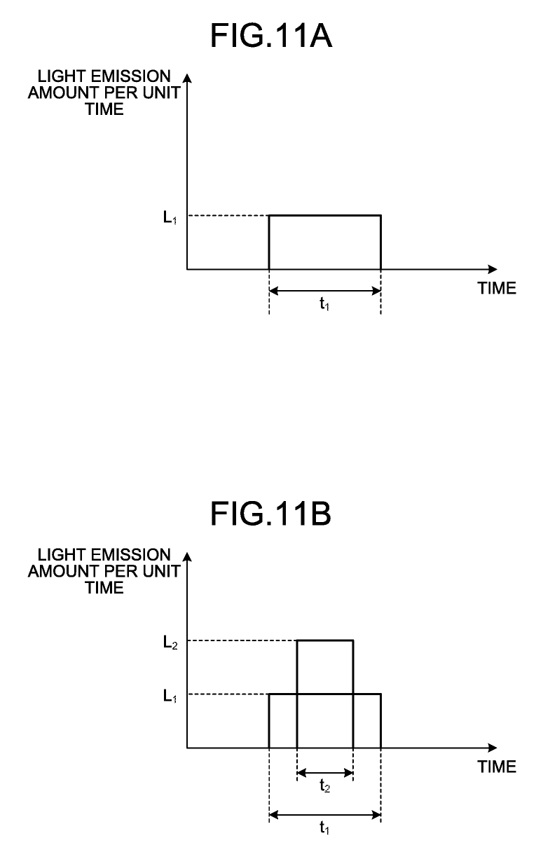

FIG. 11A is a schematic diagram illustrating the light emission amount of the illumination unit 21 in the current frame controlled by the illumination control section 292. FIG. 11B is a schematic diagram illustrating the light emission amount of the illumination unit 21 in the next frame controlled by the illumination control section 292. In FIGS. 11A and 11B, a horizontal axis represents time and a vertical axis represents the light emission amount per unit time.

As illustrated in FIGS. 11A and 11B, the illumination control section 292 shortens the light emission time in the next frame compared to the light emission time in the current frame. Besides, the illumination control section 292 performs control in such a manner that the light emission amount of the illumination light with which the illumination unit 21 performs irradiation in the next frame becomes larger than the light emission amount of the illumination light with which the illumination unit 21 performs irradiation in the current frame, such that the light receiving amount received by the imaging unit 23 becomes the same light receiving amount as that has been received within the light emission time in the current frame. Specifically, the illumination control section 292 shortens a light emission time t₂ in the next frame by half relative to a light emission time t₁ in the current frame. Furthermore, the illumination control section 292 increases the light emission amount of the illumination light in the next frame with which the illumination unit 21 performs irradiation by supplying a light emission amount L₂ per unit time to the illumination unit 21 in such a manner that the light emission amount L₂ becomes equal to or more than double the amount of a light emission amount L₁ per unit time to be supplied to the illumination unit 21 in the current frame. Accordingly, the light receiving amount received by the imaging unit 23 becomes equal to or more than the light receiving amount in the current frame even in a case where the light emission time in the next frame is shortened compared to the light emission time in the current frame. As a result, the light emission time of the illumination light with which the illumination unit 21 performs irradiation is shortened, whereby the image blurring may be prevented.

According to the fifth embodiment described above, when a third comparing portion 294g determines that image data in the current frame is not a bright image, the illumination control section 292 shortens the light emission time in the next frame compared to the light emission time in the current frame, and increases the light emission amount of the illumination light with which the illumination unit 21 performs irradiation in the next frame compared to the light emission amount of the illumination light with which the illumination unit 21 performs irradiation in the current frame such that the light receiving amount received by the imaging unit 23 becomes equal to or larger than the light receiving amount in the current frame. Therefore, the next frame is subject to proper exposure and image blurring may be prevented after detecting a motion of the capsule endoscope 2a with a high precision.

### Other Embodiments

The present disclosure is not limited to the embodiments described above, and various kinds of variations and applications are possible. For example, besides a capsule endoscope used in the description, the present disclosure may be applied to an endoscope device (flexible endoscope) provided with an imaging unit placed at a distal end of an insertion part that is insertable into a subject, a nasal endoscope device, a rigid endoscope, an imaging device, a medical device and the like, and an industrial endoscope.

In addition, in the description of each operation flowchart described above in the present specification, the operations have been described using the terms “first”, “next”, “subsequently”, “thereafter”, and the like, for convenience. However, these terms do not indicate that it is essential to carry out the operations in that order.

Each of the processing methods performed by the capsule endoscope in the embodiments described above, that is, the processing illustrated in each flowchart, may be stored as a program that may be executed by a control unit such as a CPU. Furthermore, such a program may be distributed after being stored in a storage medium of an external storage device such as a memory card (ROM card, RAM card, etc.), a magnetic disk, a hard disk, an optical disk (CD-ROM, DVD, etc.), or a semiconductor memory. The control unit such as a CPU then reads the program stored in the storage medium of the external storage device, and the operations are controlled by the read program so that the processing described above may be executed.

According to the present disclosure, an advantageous effect is afforded in that motions of the endoscope may be detected with a high precision.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A motion determining apparatus comprising
a processor comprising hardware, wherein the processor is configured to:
acquire first and second compressed data formed by compressing each of first and second images sequentially captured inside a subject by a body-insertable apparatus provided with an image sensor and an illumination device that performs irradiation with illumination light, and a parameter with regard to the illumination device at a time of capturing the first and second images with the image sensor;
calculate a difference between a data amount of the first compressed data and a data amount of the second compressed data;
compare the difference with a first threshold value;
compare the parameter with at least one reference value; and
determine whether a motion of the body-insertable apparatus is larger than a predetermined value based on comparison results of comparing the difference with a first threshold value and comparing the parameter with at least one reference value.

2. The motion determining apparatus according to claim 1, wherein
the processor is configured to determine that the motion of the body-insertable apparatus is larger than the predetermined value when the parameter is equal to or more than the reference value while the difference is equal to or more than the first threshold value.

3. The motion determining apparatus according to claim 1, wherein
the processor is configured to determine that the second image is a blurred image when the parameter is equal to or more than the reference value while the difference is equal to or more than the first threshold value.

4. The motion determining apparatus according to claim 1, wherein
the parameter is a light emission time of the illumination light with which the illumination device performs irradiation, and
the processor is configured to compare a ratio of the light emission time with a second threshold value as the reference value or a third threshold value as the reference value.

5. The motion determining apparatus according to claim 1, wherein
the processor is configured to compare the difference with a fourth threshold value after the determining portion determines that the motion of the body-insertable apparatus is larger than the predetermined value.

6. The motion determining apparatus according to claim 1, wherein
the parameter is a light emission time of the illumination light with which the illumination device performs irradiation at the time of capturing the second image, and
the processor is configured to compare the light emission time at the time of capturing the second image with a sixth threshold value.

7. A body-insertable apparatus comprising:
the motion determining apparatus according to claim 1;
the image sensor;
the illumination device;
a controller comprising hardware, wherein the controller is configured to:
compress each of the first and second images; and
increase a frame rate of the image sensor or shortens a light emission time of the illumination light when the processor determines that the motion of the body-insertable apparatus is larger than the predetermined value.

8. The body-insertable apparatus according to claim 7, wherein the controller is configured to:
compare a luminance value of the second image with a seventh threshold value; and
increase a light receiving amount of the image sensor when the luminance value of the second image is less than the seventh threshold value.

9. The body-insertable apparatus according to claim 7, wherein the controller is configured to:
compare a luminance value of the second image with a seventh threshold value; and
increase a gain of a signal generated by the image sensor when the luminance value of the second image is less than the seventh threshold value.

10. The body-insertable apparatus according to claim 7, wherein the controller is configured to:
compare a luminance value of the second image with a seventh threshold value; and
increase sensitivity of the image sensor for receiving the illumination light when the luminance value of the second image is less than the seventh threshold value.

11. The body-insertable apparatus according to claim 7, wherein the controller is configured to:
compare a luminance value of the second image with a seventh threshold value; and
increase a light emission amount of the illumination light emitted by the illumination device when the luminance value of the second image is less than the seventh threshold value.

12. A method of determining motion, comprising:
acquiring first and second compressed data formed by compressing each of first and second images sequentially captured inside a subject by a body-insertable apparatus provided with an image sensor and an illumination device that performs irradiation with illumination light, and a parameter with regard to the illumination device at a time of capturing the first and second images with the image sensor;
calculating a difference between a data amount of the first compressed data and a data amount of the second compressed data;
comparing the difference with a first threshold value;
comparing the parameter with at least one reference value; and
determining whether a motion of the body-insertable apparatus is larger than a predetermined value based on comparison results of comparing the difference with a first threshold value and comparing the parameter with at least one reference value.

13. A non-transitory computer-readable recording medium on which an executable program is recorded, the program instructing a processor to execute:
acquiring first and second compressed data formed by compressing each of first and second images sequentially captured inside a subject by a body-insertable apparatus provided with an image sensor and an illumination device that performs irradiation with illumination light, and a parameter with regard to the illumination device at a time of capturing the first and second images with the image sensor;
calculating a difference between a data amount of the first compressed data and a data amount of the second compressed data;
comparing the difference with a first threshold value;
comparing the parameter with at least one reference value; and
determining whether a motion of the body-insertable apparatus is larger than a predetermined value based on comparison results of comparing the difference with a first threshold value and comparing the parameter with at least one reference value.
